(19) United States
(12) Patent Application Publication
(10) Pub. No.: US 2025/0305947 A1
(43) Pub. Date: Oct. 2, 2025

(54) METHOD FOR IN-LINE MONITORING THE CONCENTRATION OF METAL SULFATES, IN-LINE MEASURING SYSTEM AND INSTALLATION ASSEMBLY

(71) Applicant: Endress+Hauser Group Services AG, Reinach (CH)

(72) Inventor: Hans Rönnbäck, Upplands Väsby (SE)

(21) Appl. No.: 19/092,582

(22) Filed: Mar. 27, 2025

(30) Foreign Application Priority Data
Mar. 27, 2024 (EP) 24167043.9

(51) Int. Cl. G01N 21/3577; G01N 21/33; G01N 21/359; G01N 21/65; G01N 33/18

(52) U.S. Cl. CPC G01N 21/3577; G01N 21/33; G01N 21/359; G01N 21/65; G01N 33/1813; G01N 33/182

(57) ABSTRACT

A method for in-line monitoring of the concentration of metal sulfates in a solution. The method includes: obtaining in-line a UV-VIS measuring spectrum of the solution; determining in-line the concentration of CoSO4 and NiSO4 by analyzing in-line the UV-VIS measuring spectrum; determining in-line the total metal sulfate concentration; and determining in-line the concentration of MnSO4, based at least on the determined concentration of NiSO4, the determined concentration of CoSO4, and the determined total metal sulfate concentration. Advantageous embodiments of an in-line measuring system and an installation assembly with the in-line measuring system are also disclosed.

### CROSS-REFERENCE TO RELATED APPLICATION

The present application is related to and claims the priority benefit of European Patent Application No. 24167043.9, filed on Mar. 27, 2024, the entire contents of which are incorporated herein by reference.

### TECHNICAL FIELD

The present disclosure relates to a method for in-line monitoring the concentration of individual metal sulfates, namely at least Nickel Sulfate (NiSO4), Cobalt Sulfate (CoSO4) and Manganese Sulfate (MnSO4), in a solution comprising at least one metal sulfate. The solution is contained in a pipe or a tank of an industrial plant. The present disclosure further relates to an in-line measuring system, an installation assembly, as well as the use of the installation assembly.

### BACKGROUND

The quality and/or efficiency of active cathode material production (CAM) for so-called NMC-based, NMC: short for Nickel (Ni) Manganese (Mn) and Cobalt (Co), cathode materials depends strongly on a sufficiently precise control of the composition, i.e., the concentration of the individual metal sulfates, in the feedstock preparation. NMC based materials for the positively charged cathode are commonly used in the production of lithium-ion batteries, which are, for example, used in mobile devices and/or electric vehicles. In the above-mentioned feedstock preparation process, aqueous solutions of a mixtures of at least three metals sulfates, namely NiSO4, CoSO4 and MnSO4, as well as potentially additional metal sulfates, are used. The blending of the metal sulfates relies on ratio-feedforward control, which is achieved using flow meters, for example Coriolis-type Mass flow meters, in combination with frequent manually performed grab samples, which are subsequently analyzed by means of a laboratory-type analysis, to properly adjust the measured concentrations of the metal sulfate concentrations to reach pre-set target values. Such manually performed grab samples are time consuming and costly and eventually hinder the scaling-up of the production process. Additionally, potential influences from residuals and/or impurities from upstream process steps may have an impact for feedforward ratio control of the raw materials.

Therefore, for fast and reliable process control, there is a strong need for a robust method and measuring systems which can in-line perform recurring measurements, for in-line determination and control of the concentration of individual metal sulfates. In the context of this application, “in-line measurement” relates to a measurement which is performed on-site, with measurement instruments fixedly, for example, permanently, installed in the process plant, for example in a flow-through or in a bypass of a pipe of an industrial plant. For example, such in-line measurements may be performed recurringly without interrupting the production process. An in-line measurement refers therefore to the measurement of a measurand directly and immediately on site, without, for example, any further pre-treatments of a sample of the solution being necessary. In contrast, non-in-line measurements which require such pre-treatments lead to a time delay. This means that process tracking based on a measurand not measured in-line makes it difficult to control and/or regulate a process based on measurands not measured in-line.

### SUMMARY

It is therefore the object of the present disclosure to provide a robust yet accurate in-line monitoring of the concentration of individual metal sulfates in a solution of several metal sulfates.

The object is achieved by a method for in-line monitoring of the concentration of individual metal sulfates, for example, NiSO4, CoSO4 and MnSO4, in a solution comprising at least one metal sulfate, wherein the method may be performed with an in-line measuring system, an installation assembly, as well as the use of the installation assembly.

Advantageous embodiments are specified in the present disclosure. Respective embodiments of the method are included in the measuring system and the installation assembly. The measuring system and installation assembly can also be adapted to include the method.

The object is achieved by a method for in-line monitoring of the concentration of at least NiSO4, CoSO4 and MnSO4 in a solution comprising at least one metal sulfate, wherein the solution may be contained in a pipe or tank of an industrial plant, the method comprising:
In-line obtaining a UV-VIS measuring spectrum of the solution, for example, by using ultraviolet or ultraviolet-visible or near infrared electromagnetic waves having a wavelength within a range from 200 nm to 1000 nm, preferably 300 nm to 900 nm,
In-line determining the concentration of CoSO4 as a first metal sulfate contained in the solution and the concentration of NiSO4 as a second metal sulfate contained in the solution, based at least on an in-line analysis of the UV-VIS measuring spectrum,
In-line determining the total metal sulfate concentration contained in the solution, by using at least one in-line measurement which is sensitive to the total concentration of the metal sulfates,
wherein the in-line determination of the total metal sulfate concentration is performed in addition to the in-line obtaining of the UV-VIS measuring spectrum, by using a method different from UV-VIS;
In-line determining the concentration of MnSO4 as a third metal sulfate contained in the solution, based at least on the determined concentration of NiSO4, the determined concentration of CoSO4, and the determined total metal sulfate concentration.

The UV-VIS measuring spectrum is obtained by in-line subjecting the solution to ultraviolet or ultraviolet-visible or near-infrared absorption and/or reflectance spectroscopy or spectrophotometry. This is used to determine the concentration of CoSO4 as a first metal sulfate and NiSO4 as a second metal sulfate.

The at least one in-line measurement for determining the total metal sulfate concentration is sensitive to and therefore indicative of the total metal sulfate concentration.

Depending on the specific application, the individual and total metal sulfate concentrations may be determined in terms of a volumetric concentration, molar concentration, or mass density concentration with the method according to the present disclosure.

Investigations of the applicant have demonstrated that it is feasible to use in-line UV-VIS spectroscopy or spectrophotometry, for a reliable determination the individual concentration of NiSO4 and CoSO4 in a solution containing one or more metal sulfates. The total metal sulfate concentration, i.e., the total concentration of all metal sulfates contained in the solution, is then used to indirectly determine the concentration of the third metal sulfate, namely MnSO4, which does not have a strong, identifiable absorption/reflectance signature in the UV-VIS measuring spectrum.

In the simplest case, wherein the solution comprises only NiSO4, CoSO4 and MnSO4 as metal sulfates, the MnSO4 concentration may then be determined by subtracting the NiSO4 and CoSO4 concentrations from the determined total metal sulfate concentration.

In an embodiment of the method, wherein in the industrial plant, MnSO4 is admixed to a first solution comprising NiSO4 and CoSO4 as metal sulfates, yielding a second solution,
the determining of the total mass metal sulfate concentration is carried out both with the first solution and with the second solution, and wherein the determining of the concentration of MnSO4 in the second solution is based at least on:
the determined concentration of NiSO4 of the first solution and/or the determined concentration of NiSO4 of the second solution,
the determined concentration of CoSO4 of the first solution and/or the determined concentration of CoSO4 of the second solution, and
the determined total metal sulfate concentration of the first solution and the determined total metal sulfate concentration of the second solution.

This means that the determination of the concentration of MnSO4 is based both on measurements performed before and after admixing MnSO4 to the solution.

Additionally, the measurements may also be performed before and after admixing one of the other, preferably before and after admixing each of the other metal sulfates, for a more precise determination of the total and/or individual metal sulfate concentration.

In an embodiment of the method, the at least one in-line measurement for determining the total metal sulfate concentration comprises at least one of the following:
raman spectroscopy performed with an in-line Raman spectrometer, wherein a Raman measuring spectrum is in-line analyzed to determine the total concentration of all metal sulfates in the solution,
a total mass density measurement performed with an in-line mass density measuring apparatus,
a Refractive index measurement carried out with an in-line refractometer.

Only a single one of the above in-line measurement (Raman Spectroscopy, mass density and/or Refractive index) may be used in case the solution is essentially composed of metal sulfates, wherein no significant amount of other types of molecules different from the metal sulfates and contributing to the Raman Spectrum, Mass Density, Refractive index, respectively, are present in the solution. In other cases, the determining the total metal sulfate concentration may comprise more than one of the above measurements and, potentially, the consideration of further in-line measurable process variables.

The in-line mass density measuring apparatus is, for example, embodied as a mass flow meter, for example, as Coriolis mass flow meter. The in-line mass density measuring apparatus may, for example, comprise a tuning fork as a mechanically oscillatable unit and/or a U-shaped oscillating tube for guiding the solution, and may be based on a so-called MEMS sensor, The in-line mass density measuring apparatus may also be embodied as an in-line ultrasonic mass concentration meter.

In an embodiment of the method, the solution contains a fourth metal sulfate and the atomic number of the metal of the fourth metal sulfate differs by at least 5 or at least by 10 from the atomic numbers of the metals of the first, second and third metal sulfates, namely Co, Ni and Mn, the method comprises:
performing in-line Gamma-ray or X-ray absorption measurements, for example, using a low energy gamma or X-ray source having energies below 350 keV, or below 200 keV, and obtaining a Gamma-ray or X-ray measuring spectrum,
in-line determining the concentration of the fourth metal sulfate, by analyzing the Gamma-ray or X-ray measuring spectrum, and
in-line determining the concentration of MnSO4 as a third metal sulfate contained in the solution,
based at least on the determined concentration of NiSO4, the determined concentration of CoSO4, the determined concentration of the fourth metal sulfate, and the determined total metal sulfate concentration.

In an embodiment of the method, in case the solution contains less than 500 ppm, or less than 100 ppm, of other ions which are no metal sulfates, in relation to the total ion concentration, the at least one measurement method for determining the total metal sulfate concentration an in-line conductivity measurement, carried out with an in-line conductivity sensor.

In an embodiment of the method, the method comprises:
in-line determining the concentration of H2O2 in the solution, by in-line analyzing the UV-VIS measuring spectrum and/or by in-line analyzing the oxidation reduction potential measured in-line with an in-line oxidation reduction potential sensor, and/or
in-line determining the concentration of H2SO4, by in-line measuring the pH-value, and
considering the determined H2O2 concentration and/or the determined H2O4 concentration in the solution, in the determining of the total and/or individual metal sulfate concentration of the first, second, third, and, if applicable, fourth metal sulfate.

Acid residuals such as H2SO4 and/or H2O2 may influence the determined concentrations mentioned above, for example, the ones determined indirectly such as the MnSO4 concentration. Therefore, in this embodiment, a cross-sensitivity of the determined concentrations on the acid residuals present in the solution is advantageously considered.

In an embodiment of the method, the method comprises:
In-line determining the temperature of the solution, and
Considering the determined temperature, in the determining of the total and/or individual metal sulfate concentration of the first, second, third, and, if applicable, fourth metal sulfate.

Therefore, in this embodiment, a cross-sensitivity of the determined concentrations on the temperature of the solution is advantageously considered. The determined temperature may also be used to convert volumetric concentrations into mass concentrations, or vice versa.

In an embodiment of the method, the in-line determination of the concentration of the first, the second, the third, and, if applicable, the fourth metal sulfate comprises in-line performing an analysis, for example, a multivariate analysis, wherein the analysis includes the processing of at least two of the following:
the UV-VIS measuring spectrum, the Raman measuring spectrum, the determined total mass density, the Gamma-ray or X-ray measuring spectrum, the determined pH-value, the determined conductivity, the determined oxidation reduction potential, the determined concentration of H2SO4, the determined concentration of H2O2, the determined temperature.

The analysis may be based on an analytical model to map the measuring spectrum/spectra and/or the aforementioned determined measured values to the concentrations of the metal sulfates to be determined in-line. Of course, more advanced models could be used for such a mapping, for example based on a machine learning or AI model, such as an artificial neural network. Such an artificial neural network is, for example, one of the following:
a fully connected neural network,
a single-or multilayer perceptron,
a multilayer neural network with a large number of intermediate layers,
a convolutional neural network,
a recurrent neural network.

In an embodiment of the method, a model used in the, for example, multivariate, analysis is calibrated, verified adjusted and/or trained, by using a laboratory spectrometer, for example, an inductively coupled plasma atomic emission spectroscopy spectrometer, in a laboratory located in the industrial plant or remote from the industrial plant.

Such laboratory spectrometers, which cannot be used as part of an in-line measuring system, may be advantageously used within the method according to present disclosure, to properly calibrate, verify, adjust the model, and/or, in case of a machine learning or AI model, for the training of said model.

Using a laboratory spectrometer as a reference method, reference measured values for metal sulfate concentrations are acquired. These reference values are mapped to the concentrations determined by the method according to the present disclosure by use of the model, with an acceptable correlation accuracy. This is for example carried out before the in-line determining of the individual metal sulfate concentrations, as part of an initialization process and/or repeated after a set operation time of the in-line measuring system.

The model acquired within said initialization process is then used subsequently in the method, so that the individual metal sulfate concentrations can be determined both in-line and with a sufficient accuracy required in the CAM production process.

Regarding the measuring system, the object of the present disclosure is achieved by an in-line measuring system, wherein the in-line measuring system is embodied to carry out the method according to the present disclosure, wherein the measuring systems comprises:
an in-line ultraviolet or ultraviolet-visible absorption and/or reflectance spectrometer or spectrophotometer, for in-line determining an ultraviolet-visible or ultraviolet absorption and/or reflectance spectrum,
at least on in-line measuring apparatus different from the ultraviolet or ultraviolet-visible absorption and/or reflectance spectrometer or spectrophotometer, wherein the in-line measuring apparatus is embodied to in-line determine the total metal sulfate concentration contained in the solution, by using at least one measurement method which is sensitive to the total concentration of the metal sulfates, and
an evaluation and/or computation unit, embodied to determine the concentration of at least NiSO4, CoSO4 and MnSO4 in the solution.

In an embodiment of the measuring system, the system comprises an in-line Raman spectrometer. The in-line Raman spectrometer is used to acquire the in-line Raman measuring spectrum.

As set out below, other embodiments of the measuring systems may comprise additional measuring apparatuses, for example to in-line determine one or more of the aforementioned measuring spectra (Gamma-ray or X-ray measuring spectrum) and/or to in-line determine measurands for measured variables such as the aforementioned total mass density, the pH-value, the conductivity, the oxidation reduction potential, the concentration of H2SO4, the concentration of H2O2, the temperature etc. Additional measured variables may comprise but are not limited to conductivity, viscosity, mass flow rate. For example, with a Coriolis-type mass flow meter, the mass density, viscosity, and mass flow rate are advantageously determined simultaneously.

Regarding the installation assembly, the object of the present disclosure is achieved by an installation assembly for producing a solution containing metal sulfates in an industrial plant. The installation assembly comprises:
a tank for containing the solution,
a pipe system comprising pipes for guiding the solution, wherein the pipes lead into or out of the tank, and
at least a first in-line measuring system according to the present disclosure, wherein the first in-line measuring system is installed in the pipe system.

In an embodiment of the installation assembly, the installation assembly comprises:
a first, second, third, and, if applicable, fourth supply storage for the first, second, third, and, if applicable, fourth metal sulfate, respectively,
supply pipes leading from the first, second, third, and, if applicable, fourth supply storage to the tank, for producing the solution,
wherein the installation assembly is designed in such a way that the concentration of the first, second, third, and if applicable, fourth metal sulfate in the solution is determined with at least the first in-line Measuring system, on its way from its respective supply storage into the tank, during the admixing of the respective metal sulfate to the solution,
and wherein the installation assembly may be embodied to in-line blend the first, second, third, and, if applicable, fourth metal sulfate with one another, on their way from their respective supply storage into the tank, preferably using static mixing.

In another embodiment of the installation assembly, the installation assembly comprises:
a second in-line measuring system according to the present disclosure, wherein the second in-line measuring system is arranged in a circuit pipe, wherein the circuit pipe is leading from a first outlet leading out of the tank to an inlet leading into of the tank
and/or
comprising a third in-line measuring system according to the present disclosure, wherein the third in-line measuring system is arranged with respect to a feeding pipe, which feeding pipe leads from a second outlet of the tank to a crystallizer in such a way that the concentration of the first, second, third, and if applicable, fourth metal sulfate in the solution is determined on its way from the tank to the crystallizer.

In an embodiment of the installation assembly, the installation assembly is embodied to control the inflow from the supply pipes into the tank, based on the concentration of the first metal sulfate, the second metal sulfate, the third metal sulfate, and/or, if applicable, the fourth metal sulfate, determined in-line with the first in-line measuring system, the second in-line measuring system and/or the third in-line measuring system.

For example, the first, second and/or third in-line measuring system is/are installed in a respective pipe measuring section, which pipe measuring section has a length of less than 3 m in a longitudinal direction of the pipe.

The present disclosure also relates to the use of the installation assembly according to the present disclosure, in a feeding pipe for a crystallizer for crystallization of cathode materials for lithium-ion batteries.

### BRIEF DESCRIPTION OF THE DRAWINGS

The present disclosure is explained in more detail with reference to the following figures, which are not to scale, where identical reference signs denote identical features. Where clarity so requires or it otherwise appears expedient, reference signs already mentioned are omitted in subsequent figures.

All embodiments of the method are mutatis mutandis comprised by the measuring system as well as the installation assembly, and vice versa.

The following are shown:

FIG. 1a shows a UV-VIS measuring spectrum of a solution containing metal sulfates;

FIG. 1b shows a flowchart of an embodiment of the method according to the present disclosure;

FIG. 1c shows a flowchart of a further embodiment of the method according to the present disclosure;

FIGS. 2a and 2b show schematic views of exemplary embodiments of the in-line measuring system according to the present disclosure; and

FIGS. 3a, 3b, and 3c show schematic views of exemplary embodiments of the installation assembly according to the present disclosure.

### DETAILED DESCRIPTION

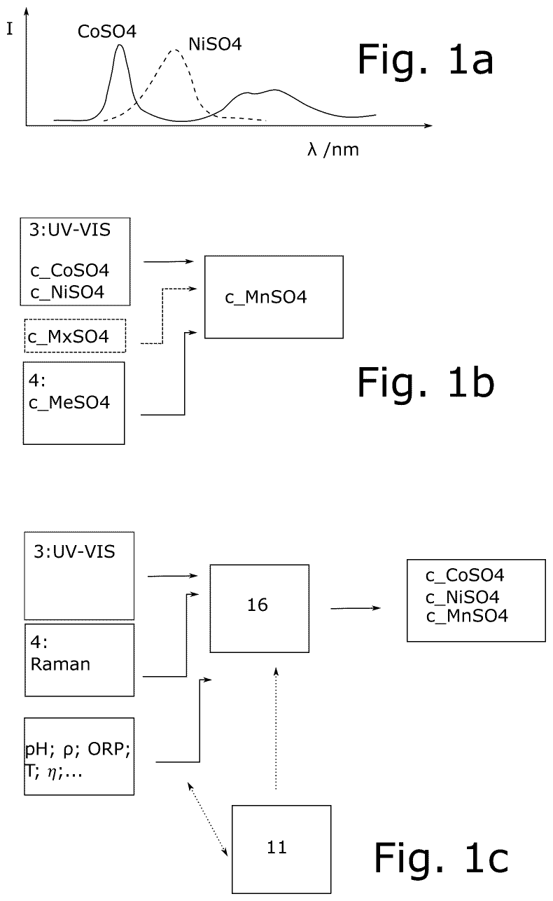

FIG. 1a shows investigations of Applicant, wherein a first UV-VIS spectrum (intensity over wavelength) of a solution containing Cobalt sulfate CoSO4 (first metal sulfate, solid line) and a second UV-VIS spectrum of a solution containing Nickel sulfate NiSO4 (second metal sulfate, dashed line) were collected. The investigations demonstrate that the first and second metal sulfate exhibit two clearly distinguishable peaks in the UV-VIS measuring spectrum, comprising a first peak (solid line) for CoSO4 and second peak for NiSO4 (dashed line).

The method according to the present disclosure makes use of this by using an in-line UV-VIS spectrometer or spectrophotometer 3, to in-line determine the concentration of the first and second metal sulfate in a solution 1, 1a, 1b contained in a pipe 2a, 2b, 2c or a tank 2 of an industrial plant.

FIG. 1b shows a flowchart of steps comprised by the method, in first embodiment of the method according to the present disclosure. First, the cobalt sulfate concentration c_CoSO4 and the nickel sulfate concentration c_NiSO4 contained in the solution 1, 1a, 1b are determined, by in-line obtaining and analyzing the UV-VIS measuring spectrum, for example, by using ultraviolet or ultraviolet-visible or near infrared electromagnetic waves having a wavelength within a range from 200 to 1000 nm.

This information is then combined with another in-line measurement, different than the UV-VIS, to determine the total metal sulfate concentration c_MeSO4. This is done in the embodiment of the method shown in FIGS. 1b and 1c by using in-line Raman spectroscopy performed with an in-line Raman spectrometer 4, wherein a Raman measuring spectrum is in-line analyzed. Without limiting the generality, the present disclosure is explained now and hereinafter in connection with an in-line Raman spectrometer 4 for the determination of the total metal sulfate concentration. The present disclosure is, however, not limited to using Raman spectroscopy and may, alternatively or additionally, comprise one of the following to determine the total metal sulfate concentration c_MeSO4:
a total mass density measurement carried out with an in-line mass density measuring apparatus 5;
a refractive index measurement performed with an in-line refractometer 6; and/or
a conductivity measurement performed with a conductivity sensor 7.

Once the total metal sulfate concentration c_MeSO4, the cobalt sulfate concentration c_CoSO4 and the nickel sulfate concentration c_NiSO4 are determined, the manganese sulfate concentration c_MnSO4 can then be determined indirectly from the other individual and the total metal sulfate concentrations. For example, wherein the solution comprises only NiSO4, CoSO4 and MnSO4 as metal sulfates, the manganese sulfate concentration c_MnSO4 may be determined by simply subtracting the nickel sulfate concentration c_NiSO4 as well as the cobalt sulfate concentration c_CoSO4 from the determined total metal sulfate concentration c_MeSO4.

Preferably, the individual and/or total metal sulfate concentrations c_NiSO4, c_CoSO4, c_MnSO4, c_MeSO4 are determined at least twice: once for a first solution 1a and once for a second solution 1b, wherein the second solution 1b is obtained by admixing manganese sulfate to the first solution 1a. Thus, total metal sulfate concentration c_MeSO4 of both the first solution 1a and the second solution 1b, before and after admixing the manganese sulfate, is determined, for example, by using the same Raman spectrometer 4 or several Raman spectrometers 4. This increases the accuracy of the method according to the present disclosure.

Of course, the total metal sulfate concentration c_MeSO4 may also be determined both before and after adding NiSO4 and/or CoSO4, to increase the accuracy of the method according to the present disclosure. Likewise, the accuracy may be increased, when several UV-VIS measuring spectra are obtained and analyzed and the cobalt sulfate concentration c_CoSO4 and nickel sulfate concentration c_NiSO4 are determined both before and after admixing several individual metal sulfates to the solution 1, 1a, 1b.

Additionally (see the optional dashed box in FIG. 1b), in case a fourth metal sulfate is present in the solution 1, 1a, 1b and the atomic number of the metal of the fourth metal sulfate differs by at least 5, for example, at least by 10 from the atomic numbers of the metals of the first, second and third metal sulfate, the concentration of that fourth metal sulfate MxSO4 is additionally determined using in-line Gammy-ray or X-ray absorption measurements.

As illustrated in FIG. 1c, the UV-VIS measuring spectrum, the Gammy-ray or X-ray measuring spectrum (if applicable) and the one or more measurements to determine the total metal sulfate concentration c_MeSO4 may be analyzed by considering one or more additional measurands determined in-line. The additional measurands collected in-line, may comprise measurands one of the following measured variables:
the pH-value, the conductivity, the oxidation reduction potential, the temperature, the viscosity, etc.

Depending on the presence of ions different from the metal sulfates in the solution 1, 1a, 1b, the in-line conductivity measurement may be itself indicative of the total metal sulfate concentration c_MeSO4, or, too, serve as an additional measured variable considered to increase the accuracy of the method according to the present disclosure.

For example, acid residuals, such as H2SO4 and or peroxide H2O2 and other impurities may be present in the solution 1, 1a, 1b, for example, from upstream process steps. Determining their amount, for example by one or more of the above-mentioned measured variables, also increases the accuracy of the method according to the present disclosure.

The temperature measurement is advantageously used for temperature compensation, for example, when the measured temperature is not stable, as some of the aforementioned physical and/or electrochemical measured variables may exhibit a strong temperature dependence.

In the embodiment shown in FIG. 1c, a model 16 is used to determine the metal sulfate concentrations c_CoSO4, c_NiSO4, c_MnSO4 from the UV-VIS measuring spectrum, the Raman measuring spectrum and the additional measured variables. The term “model” may describe an analytical and/or chemometric model, but also refers to models based on machine learning or Artificial Intelligence (AI).

To validate the model 16 used in the embodiment of the method shown in FIG. 1c, an additional laboratory spectrometer 11 is used to calibrate, verify adjust and/or train the model, preferably at least in an initialization process. The laboratory spectrometer 11 uses laboratory methods and, unlike the UV-VIS spectrometer, cannot be used as an in-line spectrometer. For example, by performing elemental analysis with an elemental laboratory spectrometer 11, the elemental metal concentrations are reliably determined with high accuracy. The determined elemental metal concentrations are then correlated with the metal sulfate concentrations c_CoSO4, c_NiSO4, c_MnSO4 determined with the in-line method according to the present disclosure, in order to calibrate, verify, adjust and/or train the model in said correlation process, in order to achieve a sufficient accuracy for method according to the present disclosure.

The laboratory spectrometer 11 is embodied, for example as an inductively coupled plasma atomic emission spectroscopy (ICP-OES) spectrometer, or an X-Ray Fluorescence (XRF) spectrometer.

The in-line determined metal sulfate concentrations c_CoSO4, c_NiSO4, c_MnSO4, . . . with the method according to the present disclosure may be advantageously used for an in-line monitoring of the feedstock preparation process for the CAM production. Preferably, recurrent in-line measurements are performed often enough to closely monitor and/or control the process. For example, at least 1 measurement value per 30 minutes, preferably at least 1 measurement value per 10 minutes, for example, at least 1 measurement value per 5 minutes is collected with the in-line measurement system 100 (see below), preferably in a periodic manner. At an optimum, an essentially continuous in-line measurement with a measurement frequency of 1 measurement per 1 minute is performed.

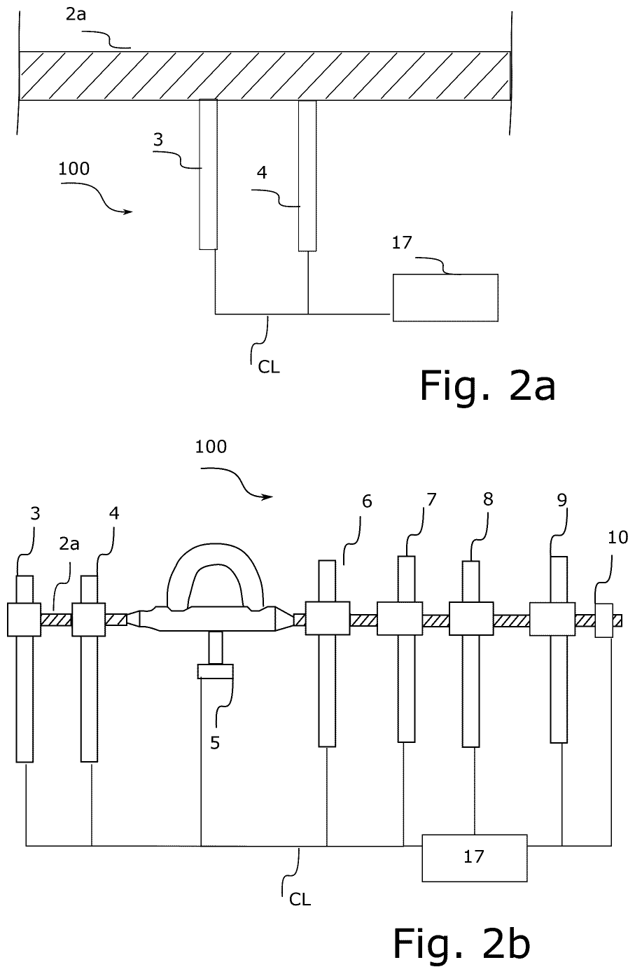

FIG. 2a shows a schematic view of a first embodiment of the measuring system 100, comprising the UV-VIS spectrometer or spectrophotometer 3 and, again, a Raman spectrometer 4 for determining the total metal sulfate concentration c_MeSO4. The measuring system 100 is installed in a pipe measuring section of a pipe 2a of an industrial plant. The pipe 2a serves for guiding the solution 1, for example, to a tank 2

The UV-VIS measuring spectrum as well as the Raman measuring spectrum are transmitted via a communication link CL to an evaluation and/or computation unit 17, which includes at least one processor. The communication link CL is, for example, embodied as a wired communication connection, a wired automation technology fieldbus, e.g., Foundation Fieldbus, Profibus PA, Profibus DP, HART, Modbus RTU, CAN bus. However, it can also be a communication connection of a modern industrial communication network, e.g., an “Industrial Ethernet” fieldbus, in particular Profinet, Ethernet/IP, Modbus TCP, HART-IP, MQTT or OPC UA, applied according to their standard specification or a field bus version, in particular the IEEE 802.3cg-based standards such as Ethernet-APL or a communication network known from the communication sector, e.g., Ethernet according to the TCP/IP protocol. If the communication connection is wireless, it can be a Bluetooth, ZigBee, WLAN, GSM, LTE, UMTS communication network or a wireless version of a field bus, in particular 802.15.4-based standards such as WirelessHART.

By using the method according to the present disclosure, the evaluation and/or computation unit 17 determines at least the nickel sulfate concentration c_NiSO4, the cobalt sulfate concentration c_CoSO4, and the manganese sulfate concentration c_MnSO4.

FIG. 2b shows a second embodiment of the measuring system 100, installed in-line in a measuring section of a pipe 2a. The measuring system 100 comprises various in-line measurement apparatuses 3, 4, 5, 6, 7, 8, 9, 10 which are in such an embodiment combined in a compact manner. For example, the pipe measuring section in which the measuring system 100 is arranged has a length of less than 3 m in a longitudinal direction of the pipe.

The measuring system 100 of the embodiment in FIG. 2b comprises the following in-line measurement apparatuses 3, 4, 5, 6, 7, 8, 9, 10:
the UV-VIS spectrometer or spectrophotometer 3,
the Raman spectrometer 4,
a mass density measuring apparatus 5,
a refractometer 6,
a conductivity sensor 7,
an ORP sensor 8,
a pH sensor 9, and
a temperature sensor 10.

The mass density measuring apparatus 5 is here and may be of the Coriolis type, which may be embodied to measure the mass density, the mass flow and the viscosity, and e.g., the temperature.

Depending on the embodiment of the method, the above-mentioned in-line apparatuses may serve for determining the total metal sulfate concentration c_MeSO4, for example, the Raman spectrometer 4 and/or the mass density measuring apparatus 5, and/or, depending on the solution 1, 1a, 1b the conductivity sensor 7.

Alternatively, the above-mentioned in-line apparatuses may serve for considering a cross-sensitivity on the determined metal sulfate concentrations of other measuring variables such as pH value and/or conductivity, by using for example the pH sensor 9 and the conductivity sensor 7, respectively.

Additionally, the above-mentioned in-line apparatuses may serve for first determining the presence of acid residuals, such as H2O2 and H2SO4, and then considering the impact of these residuals on the determined metal sulfate concentrations. For example, the same UV-VIS spectrometer or spectrophotometer 3 may be also used for the H2O2 concentration, by analyzing the same UV-VIS measuring spectrum used for determining the cobalt sulfate concentration c_CoSO4 and nickel sulfate concentration c_NiSO4. The pH sensor 9 may be used to determine the concentration of H2SO4.

Additionally, the measuring system 100 may comprise an additional in-line adsorption spectrometer (not shown), for performing in-line Gamma-ray or X-ray absorption measurements, for determining the total fourth metal sulfate concentration c_MxSO4.

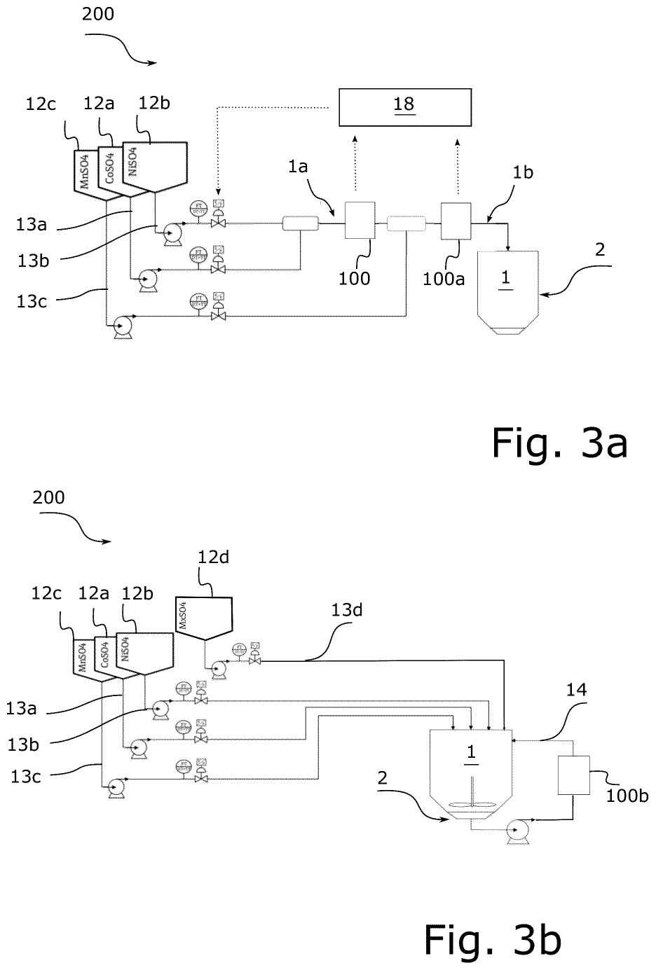

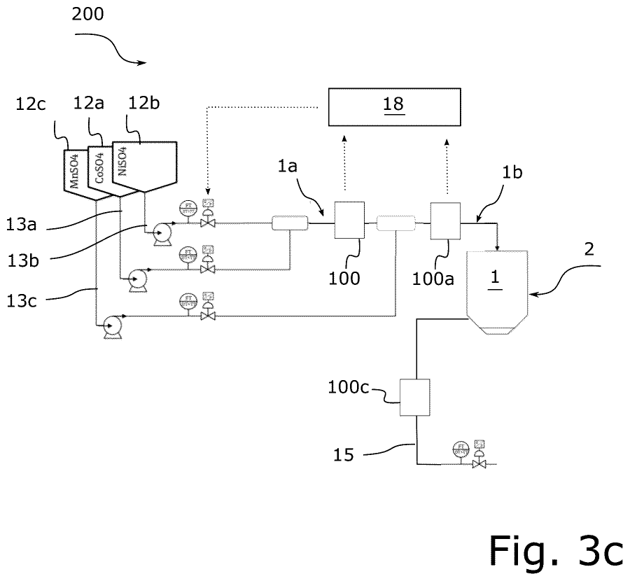

FIGS. 3a, 3b, and 3c show different embodiments of the installation assembly 200 comprising one or more of the measuring systems 100, 100a, 100b, 100c according to the present disclosure. The embodiments shown in FIGS. 3a, 3b, and 3c may be combined with one another. When comprising more than one measuring system 100, 100a, 100b, 100c, the installation assembly 200 may of course comprise different embodiments of the measuring system 100, 100a, 100b, 100c.

Starting with FIG. 3a, the installation assembly 200 may comprise supply storages 12a, 12b, 12c for the first (CoSO4), the second (NiSO4), and the third metal sulfate (MnSO4), e.g., in form of a solution containing said metal sulfate. From each of these supply storages 12a, 12b, 12c, a respective supply pipe 13a, 13b, 13c is provided for guiding the individual metal sulfate into a tank 2 containing the solution 1. The supply pipes 13a, 13b, 13c may each comprise a pump for conveying the metal sulfate, as well as a flowmeter and a valve unit, for controlling the amount of metal sulfate added to the solution 1, 1a, 1b on its way into the tank 2. Advantageously, as shown in FIG. 3a the supply pipes 13a, 13b, 13c are interconnected in such a way that the individual metal sulfates are mixed on their way from the supply storages 12a, 12b, 12c into the tank using static mixing.

A first measuring system 100 according to the present disclosure is used before the third metal sulfate MnSO4 is added, and another first measuring system 100a is used after the admixing of the third metal sulfate MnSO4. In this way, the individual metal sulfate concentrations c_NiSO4, c_CoSO4, c_MnSO4 and the total metal sulfate concentration c_MeSO4 are determined before and after adding the third metal sulfate MnSO4.

Alternatively, one single first measuring system 100a could be used to determine the individual metal sulfate concentrations c_NiSO4, c_CoSO4, c_MnSO4 and the total metal sulfate concentration c_MeSO4, both before and after admixing the third metal sulfate MnSO4, in case the installation assembly 200 comprises a topology of the pipe system suitable for this purpose, for example, by using one or more valve units.

In the embodiment shown in FIG. 3a, the individual metal sulfates are in-line mixed with one another on their way to the tank 2. For example, static mixing may be used for the in-line blending. Thus, there is no need for an additional mixer in the tank 2. Preferably, the individual metal sulfate concentrations c_NiSO4, c_CoSO4, c_MnSO4 are determined at least between each in-line mixing step.

The determined concentrations c_NiSO4, c_CoSO4, c_MnSO4 may be transmitted to a control unit 18 of the installation assembly 200, which is, for example, associated with or embodied as a Control-programmed control unit (short: SPS) of the industrial plant, to control one or more of the valves associated with the supply pipes 13a, 13b, 13c. In such a way, one or more of the in-line determined concentrations c_NiSO4, c_CoSO4 and/or c_MnSO4 may be directly used as control parameters for reaching required, preset values for the individual metal sulfate concentrations.

The second embodiment shown in FIG. 3b differs from the embodiment of FIG. 3a only slightly. A fourth metal sulfate MxSO4 contained in a fourth supply storage 12d is added to the solution 1, wherein the atomic number of the metal Mx differs by at least 5, or at least by 10 from the atomic numbers of the metals of the first, second and third metal sulfates, namely Co, Ni and Mn. Additionally, in such an embodiment, the tank 2 comprising the solution 1 may contain a mixer. In at least one embodiment, the in-line mixing shown in FIG. 3a could also be used for this embodiment, instead of or in combination with the mixer arranged in the tank 2. Similarly, the fourth metal sulfate MxSO4 and its fourth supply storage 12d may also be combined with the embodiment shown in FIG. 3a.

In this case, a second measuring system 100b is used, which is arranged in a circuit pipe 14 leading into and out of the tank 2. The circuit pipe 14 comprises also a pump for conveying the solution 1 in the circuit pipe 14. The second measuring system 100b comprises a suitable in-line adsorption spectrometer, which is embodied to perform in-line Gamma-ray or X-ray absorption measurements, for example, using a low energy gamma or X-ray source, having energies below 350 keV, or below 200 keV, and obtaining a Gamma-ray or X-ray measuring spectrum. By analyzing the Gamma-ray or X-ray measuring spectrum, the concentration of the fourth metal sulfate c_MxSO4 is determined. The manganese sulfate concentration c_MnSO4 contained in the solution 1 is, again, indirectly determined based on the nickel sulfate concentration c_NiSO4, the cobalt sulfate concentration c_CoSO4 and the total metal sulfate concentration c_MeSO4. Also, even if not explicitly shown in FIG. 3b, the determined metal sulfate concentrations c_NiSO4, c_CoSO4, c_MnSO4 and/or MxSO4, may be used, similarly to the embodiment shown in FIG. 3a, as control parameters for the installation assembly 200 of the industrial plant.

In at least one embodiment as shown in FIG. 3c, a third measuring system 100c is installed in a feeding pipe 15, which leads to a crystallizer operable for crystallizing NMC-based cathode materials. Also, the feeding pipe 15 may comprise a valve unit, whose setting depends on the metal sulfate concentrations c_NiSO4, c_CoSO4, c_MnSO4 in-line determined by the third measuring system 100c. Thus, only when the determined metal sulfate concentrations c_NiSO4, c_CoSO4, c_MnSO4 are within a specified, acceptable range, the solution 1 is then provided to the crystallizer and will be otherwise discarded or, for example, returned into the tank 2.

We claim:

1. A method for in-line monitoring of a concentration of a metal sulfate in a solution, wherein the solution is contained in a pipe or tank of a process of an industrial plant, the method comprising:
obtaining a UV-VIS measuring spectrum of the solution in-line using ultraviolet or ultraviolet-visible or near infrared electromagnetic waves having a wavelength within a range from 200 nm to 1000 nm;
determining in-line a concentration of CoSO4 in the solution as a first metal sulfate and a concentration of NiSO4 in the solution as a second metal sulfate based on at least an in-line analysis of the UV-VIS measuring spectrum;
determining a total metal sulfate concentration in the solution using at least one in-line measurement that is sensitive to the total concentration of the metal sulfates, wherein the determining of the total metal sulfate concentration is performed in addition to the obtaining and analysis of the UV-VIS measuring spectrum, and a method of the at least one in-line measurement is different from UV-VIS;
determining in-line a concentration of MnSO4 as a third metal sulfate in the solution, based at least on the determined concentration of NiSO4, the determined concentration of CoSO4, and the determined total metal sulfate concentration.

2. The method according to claim 1,
wherein in the process, MnSO4 is admixed with a first solution comprising NiSO4 and CoSO4 to yield a second solution,
wherein the determining of the total metal sulfate concentration is performed for both the first solution and the second solution and
wherein the determining of the concentration of MnSO4 in the second solution is based at least on:
the determined concentration of NiSO4 of the first solution and/or a determined concentration of NiSO4 of the second solution;
the determined concentration of CoSO4 of the first solution and/or a determined concentration of CoSO4 of the second solution; and
the determined total metal sulfate concentration of the first solution and a determined total metal sulfate concentration of the second solution.

3. The method according to claim 1, wherein the method of the at least one in-line measurement for determining the total metal sulfate concentration comprises at least one of:
a Raman spectroscopy performed with an in-line Raman spectrometer, wherein a Raman measuring spectrum is analyzed in-line to determine the total metal sulfate concentration in the solution;
a total mass density measurement performed with an in-line mass density measuring apparatus; and
a refractive index measurement performed with an in-line refractometer.

4. The method according to claim 1, wherein the solution contains a fourth metal sulfate and an atomic number of a metal of the fourth metal sulfate differs by at least 5 from atomic numbers of the metals of the first, second and third metal sulfates, the method further comprising:
performing in-line gamma-ray or X-ray absorption measurements using a low energy gamma-ray or a low energy X-ray source, each generating energies below 350 keV, and obtaining a gamma-ray or an X-ray measuring spectrum;
determining in-line a concentration of the fourth metal sulfate by analyzing the respective gamma-ray or X-ray measuring spectrum; and
determining in-line the concentration of MnSO4, as the third metal sulfate in the solution, based at least on the determined concentration of NiSO4, the determined concentration of CoSO4, the determined concentration of the fourth metal sulfate, and the determined total metal sulfate concentration.

5. The method according to claim 1, wherein when the solution contains less than 500 ppm of other ions that are not metal sulfates relative to a total ion concentration in the solution, the at least one measurement method for determining the total metal sulfate concentration comprises an in-line conductivity measurement performed with an in-line conductivity sensor.

6. The method according to claim 1, further comprising:
determining in-line a concentration of H2O2 by analyzing the UV-VIS measuring spectrum in-line and/or by analyzing an oxidation-reduction potential in-line, which is measured with an in-line oxidation reduction potential sensor, and/or determining a concentration of H2SO4 by measuring a pH-value in-line; and
considering the determined concentration of H2O2 and/or the determined concentration of H2O4 in the determining of the total metal sulfate concentration and/or individual metal sulfate concentration of the first, second, and third metal sulfate.

7. The method according to claim 1, further comprising:
determining a temperature of the solution in-line; and
applying the determined temperature in the determining of the total metal sulfate concentration and/or individual metal sulfate concentration of the first, second, and third metal sulfate.

8. The method according to claim 1, wherein the in-line determination of the concentration of the first, the second, and the third metal sulfate concentration comprises:
performing an in-line multivariate analysis, wherein the analysis includes processing of at least two of:
a UV-VIS measuring spectrum of the solution, a Raman measuring spectrum of the solution, a total mass density of the solution, a gamma-ray or X-ray measuring spectrum of the solution, a pH-value of the solution, a conductivity of the solution, a oxidation reduction potential of the solution, a determined concentration of H2SO4 of the solution, a determined concentration of H2O2 of the solution, a refractive index of the solution, and a determined temperature of the solution.

9. The method according to claim 8, wherein a model used in the multivariate analysis, which model is calibrated, verified, adjusted and/or trained using a laboratory spectrometer in a laboratory.

10. An in-line measuring system, wherein the in-line measuring system is embodied to perform the method according to claim 1, the measuring system comprising:
an in-line ultraviolet or ultraviolet-visible absorption and/or reflectance spectrometer or spectrophotometer configured to determine the UV-VIS measuring spectrum, which is at least one of an ultraviolet-visible spectrum, an ultraviolet absorption and/or a reflectance spectrum;
at least one in-line measuring apparatus different from the ultraviolet or ultraviolet-visible absorption and/or reflectance spectrometer or spectrophotometer, wherein the in-line measuring apparatus is embodied to in-line determine the total metal sulfate concentration contained in the solution by using at least one measurement method, which is sensitive to the total concentration of the metal sulfates; and
an evaluation and/or computation unit embodied to determine the concentration of at least NiSO4, CoSO4, and MnSO4 in the solution.

11. The in-line measuring system according to claim 10, further comprising an in-line Raman spectrometer.

12. An installation assembly for producing a solution containing metal sulfates in an industrial plant, the installation assembly comprising:
a tank embodied to contain the solution;
a pipe system comprising pipes configured to guide the solution, wherein the pipes lead into or out of the tank; and
a first in-line measuring system according to claim 10, wherein the first in-line measuring system is installed in the pipe system.

13. The installation assembly according to claim 12 further comprising:
a first, a second, and a third supply storage, which contain the first, second, and third metal sulfate, respectively; and
supply pipes leading from the first, second, and third supply storages to the tank to facilitate producing the solution,
wherein the installation assembly is configured such that the concentrations of the first, second, and third metal sulfates in the solution are determined with the at least one in-line measuring apparatus between the respective first, second, and third supply storage and the tank during the admixing of the respective metal sulfate to the solution, and
wherein the installation assembly is embodied to blend the first, second, and third metal sulfate with one another.

14. The installation assembly according to claim 12, further comprising a second in-line measuring system according to claim 10, wherein the second in-line measuring system is arranged in a circuit pipe, wherein the circuit pipe leads from a first outlet of the tank to an inlet of the tank, and/or further comprising a third in-line measuring system according to claim 10,
wherein the third in-line measuring system is arranged with respect to a feeding pipe that leads from a second outlet of the tank to a crystallizer such that the concentration of the first, second, and third metal sulfate in the solution is determined between the tank and the crystallizer.

15. The installation assembly according to claim 14, wherein the installation assembly is embodied to control an inflow from the supply pipes into the tank based on the concentration of the first metal sulfate, the second metal sulfate, and/or the third metal sulfate, determined in-line with the first in-line measuring system, the second in-line measuring system, and/or the third in-line measuring system.

16. The use of the installation assembly according to claim 12 in a feeding pipe for a crystallizer for crystallization of cathode materials for lithium-ion batteries.

17. The method of claim 1, wherein the obtaining a UV-VIS measuring spectrum performed by using ultraviolet or ultraviolet-visible or near infrared electromagnetic waves having a wavelength within a range of 300 nm to 900 nm.

18. The method according to claim 4, wherein the atomic number of the metal of the fourth metal sulfate differs by at least 10 from the atomic numbers of the metals of the first, second, and third metal sulfates.

19. The method according to claim 4, wherein the gamma-ray or X-ray source has an energy below 200 keV.

20. The method according to claim 9, wherein the laboratory spectrometer is an inductively coupled plasma atomic emission spectrometer.

21. The installation assembly according to claim 12, further comprising:
a fourth supply storage for a fourth metal sulfate; and
a supply pipe leading from the fourth supply storage to the tank for producing the solution,
wherein the installation assembly is designed such that the concentration of the first, second, third, and fourth metal sulfate in the solution is determined with the at least one in-line measuring apparatus between the respective supply storage and the tank during the step of admixing of the respective metal sulfate to the solution, wherein
the installation assembly is embodied to in-line blend the first, second, third, and fourth metal sulfate with one another between their respective supply storage into the tank using static mixing.

22. The method according to claim 5, wherein the solution contains less than 100 ppm of other ions that are not metal sulfates relative to the total ion concentration in the solution.

23. The installation assembly of claim 13, wherein the installation assembly is embodied to in-line blend the first, second, and third metal sulfate with one another between their respective supply storage into the tank using static mixing.
